(19) United States
(12) Patent Application Publication
(10) Pub. No.: US 2024/0277859 A1
(43) Pub. Date: Aug. 22, 2024

(54) FLOW THROUGH CATION EXCHANGE CHROMATOGRAPHY PURIFICATION PROCESSES FOR ANTIBODY DRUG CONJUGATES

(71) Applicant: Genentech, Inc., South San Francisco, CA (US)

(72) Inventors: Rachel Hendricks, San Francisco, CA (US); Matthew Henry Hutchinson, San Francisco, CA (US); Mark Frederick Fedesco, San Carlos, CA (US); Bejamin Phu Tran, San Mateo, CA (US)

(73) Assignee: Genentech, Inc., South San Francisco, CA (US)

(21) Appl. No.: 18/682,046

(22) PCT Filed: Aug. 22, 2022
(86) PCT No.: PCT/US2022/075252

§ 371 Date: Feb. 7, 2024

Related U.S. Application Data
(60) Provisional application No. 63/236,170, filed on Aug. 23, 2021.

(51) Int. Cl. A61K 47/68; C07K 1/107; C07K 1/18

(52) U.S. Cl. CPC A61K 47/68; C07K 1/1077; C07K 1/18

(57) ABSTRACT

The present invention relates to a method of developing purification processes for antibody drug conjugates using cation-exchange chromatography in flow-through mode leveraging the purification conditions of the antibody intermediate without a change to the critical quality attributes (CQA) of the ADC.

### CROSS REFERENCE TO RELATED APPLICATIONS

This application claims priority to U.S. Provisional Application No. 63/236,170 filed Aug. 23, 2021, which is incorporated herein by reference in its entirety.

### FIELD OF INVENTION

In general, the present invention relates to a method of developing purification processes for antibody drug conjugates using cation-exchange chromatography in flow-through mode. Particularly, it relates to a purification process of cysteine-targeted antibody drug conjugates using cation-exchange chromatography in flow-through mode. More particularly, it relates to a purification process of cysteine-targeted antibody drug conjugates using cation-exchange chromatography in flow-through mode leveraging the purification conditions of the antibody intermediate.

### BACKGROUND OF THE INVENTION

Antibody molecules, as part of the group of protein pharmaceuticals, are very susceptible to physical and chemical degradation. Chemical degradation includes any process that involves modification of the protein via bond formation or cleavage, yielding a new chemical entity. A variety of chemical reactions is known to affect proteins. These reactions can involve hydrolysis including cleavage of peptide bonds as well as deamidation, isomerization, oxidation and decomposition. Physical degradation refers to changes in the higher order structure and includes denaturation, adsorption to surfaces, aggregation and precipitation. Protein stability is influenced by the characteristics of the protein itself, e.g. the amino acid sequence, the glycosylation pattern, and by external influences, such as temperature, solvent, pH, excipients, interfaces, or shear rates.

Antibody-drug conjugates (ADCs) are targeted anti-cancer therapeutics designed to reduce nonspecific toxicities and increase efficacy relative to conventional small molecule and antibody cancer chemotherapy. They employ the powerful targeting ability of monoclonal antibodies to specifically deliver highly potent, conjugated small molecule therapeutics to a cancer cell. ADCs consist of a potent, small molecule drug conjugated to an antibody to allow targeted delivery to a tumor cell. The conjugation process involves a chemical reaction between an antibody and a cytotoxic drug to achieve the desired Drug-to-Antibody ratio (DAR). (R. V. J. Chari, M. L. Miller, W. C. Widdison, Antibody-Drug Conjugates: An Emerging Concept in Cancer Therapy, Ange. Chem. Int. Ed. 53 (2014) 3796-3827; P. Polakis, Pharmacological Reviews (2016), 3-19). The DAR needs to be tightly controlled since it directly impacts both safety and efficacy. The DAR also needs to be controlled to an appropriately narrow specification to ensure product consistency.

The chemical reaction step required to form the antibody-drug conjugate may require reaction conditions such as long hold times, elevated pH, solvent background, etc. that could lead to protein aggregation. As the final drug substance, the level of aggregate in the conjugate must be controlled within the required specification. In addition to the total level of aggregates, particular focus may be required specifically on product multimers, which are larger than dimers, often referred to as very high molecular weight species (vHMWS). Due to an increased risk of immunogenicity from protein aggregates, particularly with very high molecular weight species (vHMWS), a concerted effort has been made to reduce the formation of this specific aggregate species (W. Wang, S. K. Singh, N. Li, M. R. Toler, K. R. King, S. Nema, Immunogenicity of protein aggregates—concerns and realities, Int J Pharm. 431 (2012) 1-11).

The starting monoclonal antibody (mAb) intermediate is manufactured and purified to achieve a similar product quality as a standard biotherapeutic agent (A. A. Shukla, B. Hubbard, T. Tressel, S. Guhan S, D. Low, Downstream processing of monoclonal antibodies—application of platform approaches, J Chromatogr B Analyt Technol Biomed Life Sci. 848 (2007) 28-39); P. Gronemeyer, R. Ditz, J. Strube. Trends in Upstream and Downstream Process Development for Antibody Manufacturing. Bioengineering (Basel) 1 (2014) 188-212).

Purification of an antibody is typically performed using Bind-Elute chromatography or Flow-Through chromatography. Weak partitioning chromatography (Kelley, B D et al., 2008 Biotechnol Bioeng 101(3):553-566; US Patent Application Publication No. 2007/0060741) and Overload Chromatography (PCT/US2011/037977) have been used on anion exchange resins (AEX) and cation exchange (CEX) resins respectively to enhance antibody purification.

A purification process leveraging the platform processes and HTS methods is typically used for antibody purification development with cation-exchange chromatography (CEX) commonly used for aggregate and impurity removal in an antibody purification process. CEX is typically operated in bind-elute mode with a relatively low target load density (H. F. Liu, J. Ma, C. Winter, R. Bayer, Recovery and purification process development for monoclonal antibody production, mAbs, 2 (2010) 480-499).

Bind-Elute Chromatography: Under Bind-Elute chromatography the product is usually loaded to maximize dynamic binding capacity (DBC) to the chromatography material and then wash and elution conditions are identified such that maximum product purity is attained in the eluate. A limitation of Bind-Elute chromatography is the restriction of the load density to the actual resin DBC. Hence Bind-Elute chromatography purification requires larger column sizes due to lower load densities. Bind-Elute mode purification steps are more complicated to develop and implement at manufacturing stage. Pooling criteria for the Bind-Elute purification step could be a critical parameter and can lead to lower yield and facility fit challenges.

Flow Through Chromatography: Using Flow Through chromatography, load conditions are identified where impurities strongly bind to the chromatography material while the product flows through. Flow Through chromatography allows high load density for standard antibodies.

Overload Chromatography: In this mode of chromatography the product of interest is loaded beyond the dynamic binding capacity of the chromatography material for the product, thus referred to as overload. The mode of operation has been demonstrated to provide antibody purification with cation exchange (CEX) media and particularly with membranes. However, a limitation of this approach is that there could be low yields with resin as there is no elution phase. Additional challenges for Overload Chromatography are appropriate critical process parameters including facility for high titer as well as proper load conditions.

High-throughput Screening (HTS) robotic equipment typically used for purification development of standard monoclonal antibodies. However, such High-throughput Screening (HTS) robotic equipment may not be suitable or safe for handling cytotoxic compounds (J. L. Coffman, J. F. Kramarczyk, B. D. Kelley, High-throughput screening of chromatographic separations: I. Method development and column modeling. Biotechnol. Bioeng., 100 (2008) 605-618; M. I. Hensgen, B. Stump, Safe Handling of Cytotoxic Compounds in a Biopharmaceutical Environment. In: Ducry L. (Eds.), Antibody-Drug Conjugates. Methods in Molecular Biology (Methods and Protocols), 1045 (2013); Humana Press, Totowa, N J. 2013, pp. 130-142). This creates challenges for use of HTS for purification of ADC's.

If aggregate formation cannot be robustly controlled during the conjugation reaction to form the ADC, a purification step must be implemented post-conjugation to achieve the drug substance aggregate specification. The purification must be done without compromising on the safety requirements of handing the potent compounds and without impacting the desired ADC product qualities such as the DAR and drug load distribution. ADC purification techniques also employ hydrophobic interaction chromatography (HIC). HIC is a useful tool for separating molecules based on their hydrophobicity. Generally, sample molecules in a high salt buffer are loaded on the HIC column. The salt in the buffer interacts with water molecules to reduce the solvation of the molecules in solution, thereby exposing hydrophobic regions in the sample molecules, which are consequently adsorbed on the HIC column. The more hydrophobic the molecule, the less salt needed to promote binding. Usually, a decreasing salt gradient is used to elute samples from the column. As the ionic strength decreases, the exposure of the hydrophilic regions of the molecules increases and molecules elute from the column in order of increasing hydrophobicity. Sample elution may also be achieved by the addition of mild organic modifiers or detergents to the elution buffer. HIC is reviewed in Protein Purification, 2d Ed., Springer-Verlag, New York, pgs 176-179 (1988).

However, HIC techniques which are generally performed at about neutral pH in the presence of high salt concentrations may lead to precipitation of the ADC as well as safety issues in handling the potent compounds due to filter fouling. (Becker C. L., Duffy R. J., Gandarilla J., Richter S. M. (2020) Purification of ADCs by Hydrophobic Interaction Chromatography. In: Tumey L. (eds) Antibody-Drug Conjugates. Methods in Molecular Biology, vol 2078. Humana, New York, NY. https://doi.org/10.1007/978-1-4939-9929-3_19)

In general, ADC purification development is more challenging due to the safety requirements of handing the cytotoxic compounds. The large-scale, cost-effective purification of ADC to sufficient purity for use as a human therapeutic remains a formidable challenge.

Various ADC purification techniques have been explained in literature. For example, CN104208719 describes elution and overload for ADC purification. However, CN104208719 does not provide any teaching for purification in flow-through mode. Further, CN104208719 does not discuss antibody purification nor provides any teaching for leveraging the purification conditions developed during antibody intermediate purification for purification of the ADC. In another example, US2013245139 uses a CEX membrane for flow through aggregate purification of the antibody. However, US2013245139 does not provide any teaching for the purification of the ADC, especially for leveraging the purification conditions developed during antibody intermediate purification for purification of the ADC.

It is therefore an object of the present invention to provide for a method of developing purification processes for antibody drug conjugates using cation-exchange chromatography in flow-through mode leveraging the purification conditions of the antibody intermediate.

It is also an object of the present invention to identify conditions during purification to remove the aggregate which also do not impact the critical quality attributes (CQA) of the ADC product such as the DAR and drug load distribution.

Another object of the present invention to provide for development of a quick and robust purification step for antibody drug conjugates using cation-exchange chromatography in flow-through mode.

A further object of the present invention to provide for a low cost, robust purification step for antibody drug conjugates using cation-exchange chromatography in flow-through mode.

It is also an object of the present invention to specifically remove the vHMWS.

The simplified ADC purification method and the ADC purification process significantly reduced the vHMWS, consistently achieved high yields and did not change critical quality attributes (CQA) of the ADC product. This purification approach can also be used to develop purification processes for vHMWS removal for ADCs with minimal development.

### SUMMARY

The present invention provides a method for leveraging purification conditions developed during antibody intermediate purification for purification of the ADC.

In one aspect the invention provides for an improved method for reducing the concentration of very high molecular weight species (vHMWS) in cysteine-directed antibody drug conjugate (ADC), the method comprising the steps;
a. performing a first purification of an antibody with cation exchange chromatography material using a first set of purification condition to obtain a purified antibody intermediate;
b. conjugating the said purified antibody intermediate with a cytotoxic agent to form a crude preparation comprising of cys ADC and protein aggregates; and
c. performing a second purification of said crude with cation exchange chromatography material in flow-through mode using said first set of purification condition to generate a purified cys ADC, wherein the said set of purification condition comprises load density, buffer species, pH and conductivity of the buffer systems.

In another aspect, the invention provides for reducing the concentration of the vHMWS in the eluate by at least 85% relative to the concentration of protein aggregates in the crude mixture of cys ADC and protein aggregates without a change to critical quality attributes (CQA) of the cys ADC.

In yet another aspect, the invention provides for a method wherein the vHMWS in the ADC is reduced to less than 0.1%.

In a yet another aspect, the invention provides for a method wherein the resin used in the cation exchange column is selected from POROS 50HS, POROS XS, and SPFF resins.

In a further aspect, the invention provides for a method of purifying cysteine-directed antibody drug conjugate (cys ADC), the method comprising the steps;
a. performing a first purification of an antibody with cation exchange chromatography material using a first set of purification condition to obtain a purified antibody intermediate;
b. conjugating the said purified antibody intermediate with a cytotoxic agent to form a crude preparation of cys ADC; and
c. performing a second purification of said crude preparation with cation exchange chromatography material in flow-through mode using said first set of purification condition to generate a purified cys ADC,
   wherein the said set of purification condition comprises load density, buffer species, pH and conductivity of the buffer systems

In one embodiment of the method of purifying the cys ADC, the cytotoxic molecule is selected from a group consisting of auristatins, maytansinoids, and DNA-damaging agents.

In another embodiment of the method of purifying the cys ADC, the DNA-damaging agents are derivatives selected from a group consisting Calicheamicin, Anthracyclines, and Pyrrolobenzodiazepines.

In a still further embodiment of the invention, the antibody used for the formation of the cys ADC is purified in a bind-elute mode.

In another embodiment of the invention, the first purification to obtain a purified antibody intermediate involves step elution.

In a yet another embodiment of the invention, the first purification to obtain a purified antibody intermediate involves gradient elution.

In another embodiment of the method of purifying the antibody, the screening method to determine the binding behavior of the antibody is High-throughput screening (HTS).

In another embodiment of the invention, the HTS employed in the purification of the antibody is used to map the binding behavior of antibody as a function of pH and Counterion concentration.

In another embodiment, the antibody HTS results which are used to map the binding behavior of antibody are leveraged to identify the flow-through conditions for ADC purification.

In a further embodiment of the invention, the protein aggregate species removed during the purification of the antibody or the cys ADC includes very high molecular weight species (vHMWS) and high molecular weight species (HMWS) of the antibody or the cys ADC.

In a still further embodiment of the invention, the protein aggregate species removed during the purification is very high molecular weight species (vHMWS).

In another embodiment of the invention, the cys ADC is selected from a site-specific conjugate via an engineered cysteine and interchain-cysteine conjugate that target native cysteines.

In another embodiment, the cys ADC is a site-specific conjugate via an engineered cysteine.

In yet another embodiment, the cys ADC is interchain-cysteine conjugate that target native cysteines.

In a still further embodiment of the invention, the method of developing purification process comprises thiomab antibiotic antibody conjugate (AAC).

In another embodiment of the invention, the pooling criteria of the cys ADC is from 0.5 to 0.5 OD.

### BRIEF DESCRIPTION OF THE DRAWINGS

FIG. 1 depicts an example of a chromatogram of the Protein impurities which were analyzed by SEC-HPLC using a TSKgel G3000SWxL column (7.8×300 mm, Tosoh Bioscience, Tokyo, Japan). The peaks were resolved with isocratic separation using a mobile phase of 15% IPA and 85% 0.2 M potassium phosphate, 0.25 M potassium chloride, pH 6.95. The flow rate was maintained at 0.5 mL/min at ambient temperature and the UV detection at 280 nm. The two main aggregate species that were detected include the vHMWS and HMWS. The HMWS is a protein dimer of Antibody-drug conjugate while the vHMWS is an oligomer of Antibody-drug conjugate.

FIG. 2 depicts an example of the average DAR and drug load distribution determined using an analytical hydrophobic interaction chromatography (HIC) method for interchain-cysteine conjugates.

FIG. 3 depicts an example of the average DAR and drug load distribution determined using an analytical hydrophobic interaction chromatography (HIC) method for site-specific conjugates.

FIG. 4 depicts an example of the Batch binding contour plots comparing the binding behavior for an antibody and its corresponding cysteine-directed antibody drug conjugate (cys ADC) on the CEX resin.

FIG. 5 depicts an example of aggregate species (vHMWS and HMWS) breakthrough of the cysteine-directed antibody drug conjugate for CEX column with a conjugate load density of 500 g/Lᵣ

### DETAILED DESCRIPTION OF THE INVENTION

The present invention relates to a method of developing purification and a process for purification of cysteine-directed antibody drug conjugates comprising purifying the antibody and leveraging the binding behavior of the antibody intermediate aggregate species for ADC purification.

For the purposes of this specification and the claims, unless otherwise indicated, all numbers expressing quantities of ingredients, percentages or proportions of materials, reaction conditions, and other numerical values used in the specification and claims, are to be understood as being modified in all instances by the term “about” whether or not explicitly indicated. The term “about” generally refers to a range of numbers that one would consider equivalent to the recited value. In many instances, the term “about” may include numbers that are rounded to the nearest significant figure. Moreover, all ranges disclosed herein are to be understood to encompass all sub ranges subsumed therein.

All publications, patents and patent applications cited herein, are hereby incorporated by reference in their entirety.

Before describing the present invention in further detail, a number of terms will be defined. Use of these terms does not limit the scope of the invention but only serve to facilitate the description of the invention.

### Definitions

The term “antibody” is used in the broadest sense and specifically covers intact monoclonal antibodies (mAb's), polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, generally the antigen binding or variable region of the intact antibody. Examples of antibody fragments include Fab, Fab′, F(ab′)₂, and Fv fragments; diabodies; linear antibodies; single-chain antibody molecules; and multispecific antibodies formed from antibody fragments.

As used herein, “antibody intermediate” refers to a purified antibody purified by performing a first purification using a first set of purification condition, which is used for conjugating with a cytotoxic agent to generate antibody drug conjugate.

As used herein and in the appended claims, the singular forms “a,” “or,” and “the” include plural referents unless the context clearly dictates otherwise.

As used herein, “binding behavior” refers to the binding or unbinding of an antibody, antibody intermediate or the ADC to the resin at specific conditions including pH and counterion concentration.

As used herein, “buffer” refers to a buffered solution that resists changes in pH by the action of its acid-base conjugate components. The buffer for the CEX chromatography aspect of this invention has a pH in a range of about 4.5-6.5, preferably about 5.3-5.7. Examples of buffers that will control the pH within this range include phosphate, acetate, citrate or ammonium buffers, or more than one. The preferred such buffers are acetate, citrate and ammonium buffers, most preferably Sodium acetate buffers. The “loading buffer” is that which is used to load the mixture of the ADC and impurities/contaminants on the CEX column and the “equilibration/wash buffer” is that which is used to wash the ADC from the column to recover the antibody while the impurities/contaminants are retained on the column. Often the loading buffer and equilibration/wash buffer will have the same pH and/or conductivity conditions.

The term “sequential” as used herein with regard to chromatography refers to having a first chromatography followed by a second chromatography. Additional steps may be included between the first chromatography and the second chromatography.

The term “continuous” as used herein with regard to chromatography refers to having a first chromatography material and a second chromatography material either directly connected or some other mechanism, which allows for continuous flow between the two chromatography materials.

The term engineered cysteine as used herein refers to antibodies with engineered reactive cysteine residues for site-specific conjugation and display homogeneous conjugates.

The term cysteine-directed antibody drug conjugate (cys ADC) as used herein refers to conjugates of an antibody with cysteine residues available for conjugation with cytotoxic agent.

The term native cysteines as used herein refers to the interchain disulfide bonds and are generated by partial reduction resulting in heterogenous conjugates comprised of 0, 2, 4, 6, and 8-DAR forms.

The term “cytotoxic agent” as used herein refers to a substance that inhibits or prevents a cellular function and/or causes cell death or destruction. Cytotoxic agents include, but are not limited to, Auristatins, Maytansinoids, and DNA-damaging agents including Calicheamicin, Anthracyclines, and Pyrrolobenzodiazepines, radioactive isotopes; chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents.

The term “DAR” as used herein is the average drug-to-antibody ratio. DAR directly impacts the safety and efficacy of the ADC and is directly controlled during the ADC manufacturing process.

The term “drug load distribution” as used herein refers to the number of drugs conjugated to the antibody.

The term critical quality attributes (CQA) includes the average drug-to-antibody ratio (DAR) and drug loading distribution and determines the amount of cytotoxic agent that can be delivered by the ADC.

The “dynamic binding capacity” of a chromatography material is the amount of product, e.g. polypeptide, the material will bind under actual flow conditions before significant breakthrough of unbound product occurs.

“Partition coefficient”, Kₚ, as used herein, refers to the molar concentration of product, e.g. polypeptide, in the stationary phase divided by the molar concentration of the product in the mobile phase.

“Loading density” refers to the amount, e.g. grams, of composition put in contact with a volume of chromatography material, e.g. liters. In some examples, loading density is expressed in g/Lₜ.

The terms “ion-exchange” and “ion-exchange chromatography” as used herein refer to a chromatographic process in which an antibody or antibody drug conjugate of interest interacts or does not interact with a charged compound linked to a solid phase ion exchange material such that impurities or aggregates in the mixture elutes from a column of the ion exchange material faster or slower than the antibody or antibody drug conjugate of interest are bound to or excluded from the resin relative to the impurities or aggregates. “Ion-exchange chromatography” specifically includes cation exchange, anion exchange, and mixed mode ion exchange chromatography.

The term “anion exchange resin” or “AEX” as used herein refers to a solid phase which is positively charged, e.g., having one or more positively charged ligands, such as quaternary amino groups, attached thereto. Commercially available anion exchange resins include DEAE cellulose, QAE SEPHADEX™ and FAST Q SEPHAROSE™ (Pharmacia). Anion exchange chromatography can bind the target molecule followed by elution or can predominately bind the impurities while the target antibody or antibody drug conjugate “flows through” the column.

Cation exchange chromatography material is a solid phase that is negatively charged and has free anions for exchange with cations in an aqueous solution (such as a composition comprising an antibody and an impurity) that is passed over or through the solid phase. In some embodiments of any of the methods described herein, the cation exchange material may be a membrane, a monolith, or resin. In some embodiments, the cation exchange material may be a resin. The cation exchange material may comprise a carboxylic acid functional group or a sulfonic acid functional group such as, but not limited to, sulfonate, carboxylic, carboxymethyl sulfonic acid, sulfoisobutyl, sulfoethyl, carboxyl, sulphopropyl, sulphonyl, sulphoxyethyl, or orthophosphate. In some embodiments of the above, the cation exchange chromatography material is a cation exchange chromatography column. In some embodiments of the above, the cation exchange chromatography material is a cation exchange chromatography membrane. Examples of cation exchange materials including resins are known in the art include, but are not limited to Mustang® S, Sartobind® S, SO₃ Monolith (such as, e.g., CIM®, CIMmultus® and CIMac® SO₃), S Ceramic HyperD®, Poros® XS, Poros® HS 50, Poros® HS 20, sulphopropyl-Sepharose® Fast Flow (SPSFF), SP-Sepharose® XL (SPXL), CM Sepharose® Fast Flow, Capto™ S, Fractogel® EMD Se Hicap, Fractogel® EMD SO₃, or Fractogel® EMD COO. In some embodiments, the cation exchange chromatography is performed in “bind-elute” mode. In some embodiments, the cation exchange chromatography is performed in “flow through” mode. In some embodiments of the above, the cation exchange chromatography material is in a column. In some embodiments of the above, the cation exchange chromatography material is in a membrane.

“Impurities” refer to materials that are different from the desired polypeptide product. The impurity may refer to product-specific polypeptides such as one-armed antibodies and misassembled antibodies, antibody variants including basic variants and acidic variants, and aggregates. Other impurities include process specific impurities including without limitation: host cell materials such as host cell protein (HCP); leached Protein A; nucleic acid; another polypeptide; endotoxin; viral contaminant; cell culture media component, etc. In some examples, the impurity may be an HCP from, for example but not limited to, a bacterial cell such as an E. coli cell (ECP), an insect cell, a prokaryotic cell, a eukaryotic cell, a yeast cell, a mammalian cell, an avian cell, a fungal cell. In some examples, the impurity may be an HCP from a mammalian cell, such as a CHO cell, i.e., a CHO cell protein (CHOP). The impurity may refer to accessory proteins used to facilitate expression, folding or assembly of multispecific antibodies; for example, prokaryotic chaperones such as FkpA, DsbA and DsbC.

High molecule weight Substance (HMWS) as used herein refers to a protein dimer of the ADC or a protein dimer of the antibody.

Very High molecule weight Substance as used herein refers to an oligomer of ADC or an oligomer of the antibody.

The term Protein as used herein includes antibody and ADC.

Purity is a relative term and does not necessarily mean absolute purity. The terms “purifying,” “separating,” or “isolating,” as used interchangeably herein, refer to increasing the degree of purity of a desired molecule from a composition or sample comprising the desired molecule and one or more impurities. Typically, the degree of purity of the desired molecule is increased by removing (completely or partially) at least one impurity from the composition.

Purification conditions is also a relative term and these conditions may vary for every purification method. Purification conditions may include load density, buffer species, pH and conductivity of the buffer systems.

### Materials & Methods

a. Drug Conjugates

Conjugation is a multi-step process to modify the protein that may differ based on the conjugate design. Purification was explored with two types of conjugates: site-specific conjugates via an engineered cysteine and interchain-cysteine conjugates that target native cysteines.

For site-specific conjugates, the purified intermediate is incubated with reductant overnight to fully reduce the native and engineered cysteines of the antibody and remove all cysteine or glutathione caps from the engineered cysteines. The reduced antibody is buffer exchanged to clear residual reductant as well as the cap species. The interchain disulfide bonds are reformed via a reoxidation step, leaving the engineered cysteines available for conjugation with the linker-drug. Excess linker-drug is added to ensure complete conjugation to all free thiols (J. Junutula, H. Raab, S. Clark, et al. Site-specific conjugation of a cytotoxic drug to an antibody improves the therapeutic index, Nat Biotechnol 26 (2008) 925-932). Depending on the linker-drug, conjugation is either quenched or halted by decreasing the pH of the reaction. Finally, the residual free drug is removed.

For interchain-cysteine conjugates, the native cysteines of the antibody intermediate are partially reduced with a pre-defined amount of reductant prior to conjugation with the linker-drug (M. M. C. Sun, K. S. Beam, C. G. Cerveny, K. J. Hamblett, R. S. Blackmore, M. Y. Torgov, F. G. M. Handley, N.C. Ihle, P. D. Senter, S. C. Alley, Reduction-Alkylation Strategies for the Modification of Specific Monoclonal Antibody Disulfides, Bioconjugate Chemistry 16 (2005) 1282-1290). Excess linker-drug is quenched and residual free drug is removed.

b. Conjugate Column Purification

Column chromatography experiments were performed using an AKTA Explorer 100 with various sizes of column packed with cation-exchange resin. The adjusted load was applied onto an equilibrated column at various load densities and the flow-through was collected. After the load phase, the column was washed with the equilibration buffer to increase cys ADC recovery. Pooling was terminated at the end of the wash phase or when the OD was <0.5. The column was regenerated with 0.5 N NaCl, sanitized with 0.5 N NaOH and stored in 0.1 N NaOH.

c. Antibody Intermediate Purification Development

High-throughput screening (HTS) was performed for the antibody intermediate using known process (P. McDonald, B. Tran, C. R. Williams, M. Wong, T. Zhao, B. D. Kelley, P. Lester, The rapid identification of elution conditions for therapeutic antibodies from cation-exchange chromatography resins using high-throughput screening, J Chromatogr A. 1433 (2016) 66-74). The HTS maps the binding behavior of antibodies as a function of pH and buffer concentration. (J. L. Coffman, J. F. Kramarczyk, B. D. Kelley, High-throughput screening of chromatographic separations: I. Method development and column modeling. Biotechnol. Bioeng., 100 (2008) 605-618). HTS with 96-well filter plates using a Tecan Robotic liquid-handling system or multi-channel pipettes was used for batch-binding experiments to develop binding and elution conditions on cation-exchange chromatography resin. Packed-bed lab-scale columns were used to confirm and optimize the conditions.

The antibody intermediate purification process implemented CEX for aggregate and host cell impurity removal and is operated in bind-elute mode at a load density≤100 g/Lᵣ (H. F. Liu, B. McCooey, T. Duarte, D. E. Myers, T. Hudson, A. Amanullah, R. van Reis, B. D. Kelley, Exploration of overloaded cation exchange chromatography for monoclonal antibody purification, J Chromatogr A. 1218 (2011) 6943-52). The product was loaded and the column was washed prior to eluting the monomer with elution buffer. The binding behavior of the antibody intermediate aggregate species was leveraged for ADC purification development.

d. Analytical Methods

The concentration of protein was quantified by UV-vis spectrophotometry (Agilent 8453). Protein concentration was determined by absorbance at 280 nm with absorbance at either 320 nm or 400 nm subtracted to correct for light scattering. The extinction coefficient, ε, of the samples was used with the equation below where I is the sample path length, and A₂₈₀ and A₃₂₀ are the measured absorbance values at 280 and 320 nm, respectively.

$$Protein concentration = \frac{{A}_{280} - {A}_{320}}{ε \times l} \times dilution factor$$

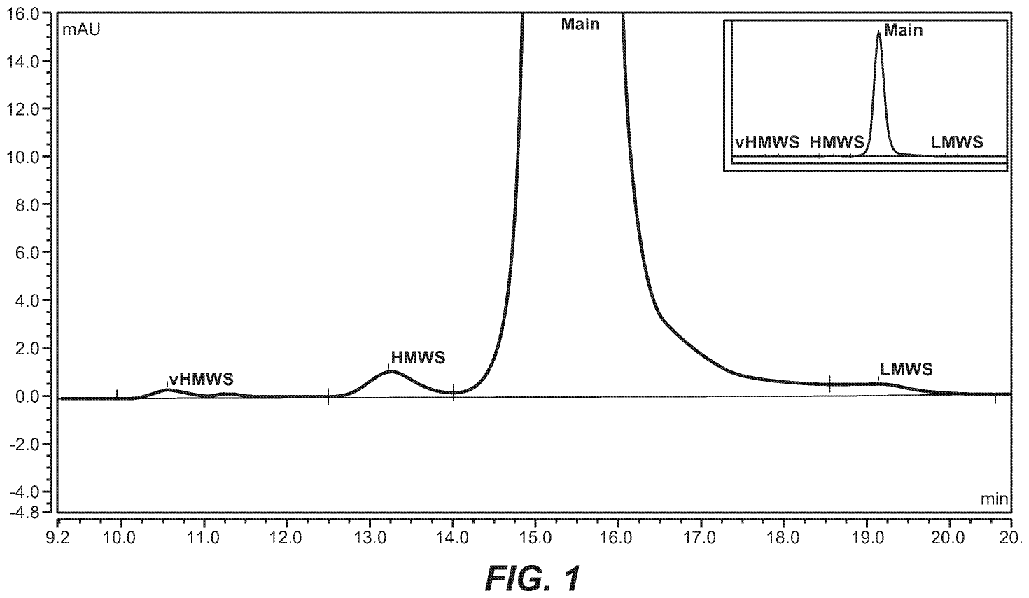

Protein impurities were analyzed by SEC-HPLC using a TSKgel G3000SWxL column (7.8×300 mm, Tosoh Bioscience, Tokyo, Japan). The peaks were resolved with isocratic separation using a mobile phase of 15% IPA and 85% 0.2 M potassium phosphate, 0.25 M potassium chloride, pH 6.95. The flow rate was maintained at 0.5 mL/min at ambient temperature and the UV detection at 280 nm. An example chromatogram is shown in FIG. 1, and is representative for both conjugate types. The two main aggregate species that were detected include the vHMWS and HMWS. The HMWS is a protein dimer while the vHMWS is an oligomer of antibody/ADC.

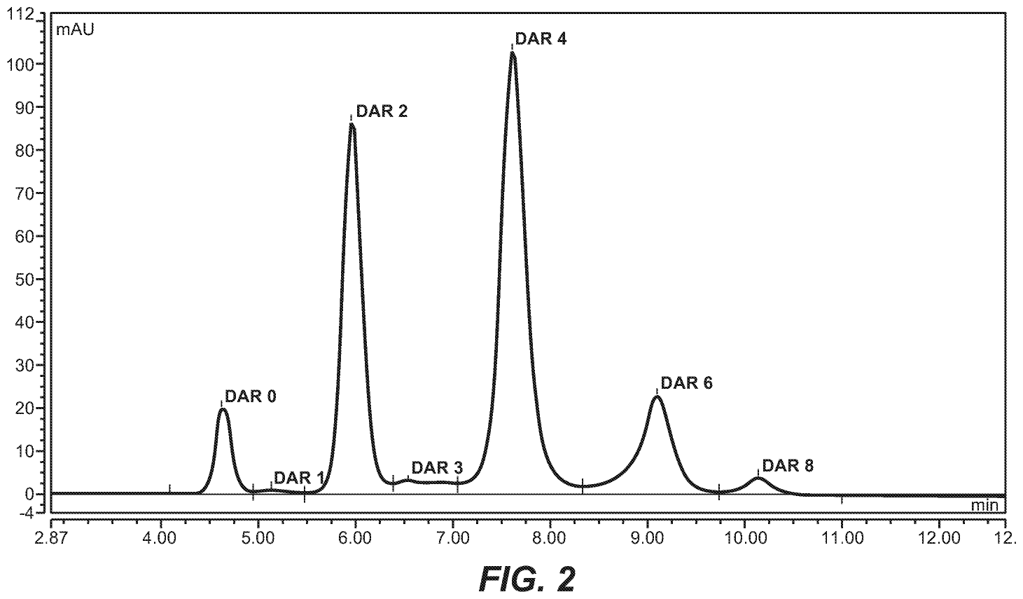

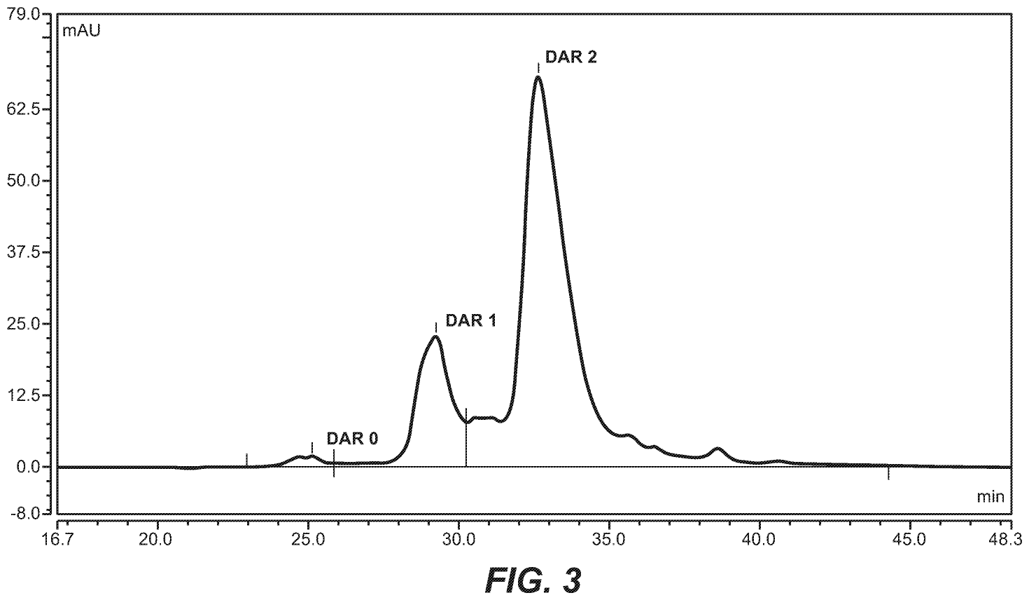

The average DAR and drug load distribution were determined using an analytical hydrophobic interaction chromatography (HIC) method as shown in FIGS. 2 and 3. Samples were injected onto the Tosoh Bioscience Butyl-NPR column (4.6 mm×3.5 cm, 2.5 μm) and eluted over a linear gradient with Solvent B at a flow rate of 0.8 mL/min with the absorbance monitored at 280 nm. Gradient and Solvent B for the individual conjugates is shown in Table 1.

TABLE 1
HIC-HPLC method gradient and solvent B

| Molecule | Gradient, % B | Solvent B |
|---|---|---|
| ADC-1 | 10-85%, 12 | 20% IPA with 25 mM |
| | minutes | Sodium Phosphate pH |
| | | 5.30 |
| ADC-2 | 10-45%, 60 | 25% IPA with 25 mM |
| | minutes | Sodium Phosphate pH |
| | | 6.95 |
| ADC-3 | 0-100%, 15 | 25% IPA with 25 mM |
| | minutes | Sodium Phosphate pH |
| | | 6.95 |

The purification processes for the ADCs were developed based on the development of their respective antibody intermediates. Each of the antibody intermediates underwent independent purification process development based on their properties (e.g., pI, binding characteristics, etc.), which resulted in slightly different purification processes and modes of operation (Table 2). Based on the different antibody purification steps, different approaches were used to develop the flow-through purification conditions for the conjugated molecules, as described herein.

ADC-1: Since Antibody-1 utilized a gradient elution, a manual resin screening was performed with ADC-1 and compared to the HTS results for Antibody-1. Promising conditions including conditions with a Log Kₚ(antibody) between 0.75-1.25 and pH/conductivity conditions that tightly bound the aggregate but not the monomer such that the monomer flows through the column were selected. The promising conditions were tested with packed-bed column experiments to determine the optimal flow-through conditions.

ADC-2: HTS was not performed for the ADC, and instead the Antibody-2 development HTS results were leveraged. The antibody intermediate step elution conditions were developed so that monomer is eluted from the column while aggregates are retained. These conditions were applied to the conjugate such that the column load material would result in product flow-through while removing aggregate species. The robustness of the load conditions on the purification capabilities were assessed using packed-bed column experiments and evaluated the performance at manufacturing scale.

ADC-3: The theory that antibody purification conditions can be applied to the ADC was tested with a third product. Similar to Antibody-2, HTS was not performed for ADC-3 and the step elution conditions from Antibody-3 were applied to the ADC to remove the aggregate species with flow-through. A single packed-bed column experiment was performed to confirm the purification capabilities for the conjugate.

TABLE 2
Molecule details and CEX purification conditions.

| | Antibody-1/ | Antibody-2/ | Antibody-3/ |
|---|---|---|---|
| Molecule | ADC-1 | ADC-2 | ADC-3 |
| Antibody CEX | Gradient | Step elution | Step elution |
| Elution Mode | elution | | |
| Antibody pI | 8.4 | 7.1 | 7.4 |
| Conjugation | Interchain- | Site-specific | Interchain- |
| chemistry | cysteine | | cysteine |
| Approximate | 0.8% | 0.5-0.8% | 0.4% |
| vHMWS in ADC, | | | |
| CEX Load | | | |

Unless otherwise stated, 0.66 cm inner diameter (ID)×1.0 cm bed height (BH) small-scale columns were used for the ADC purification runs. Also, the equilibration/wash buffer conditions were adjusted to match the load conditions. The focus of the ADC purification was vHMWS removal but HMWS removal was also monitored. Additionally, there was no desire to change the DAR or drug load distribution over the purification step.

### Example 1: ADC-1 Purification Method

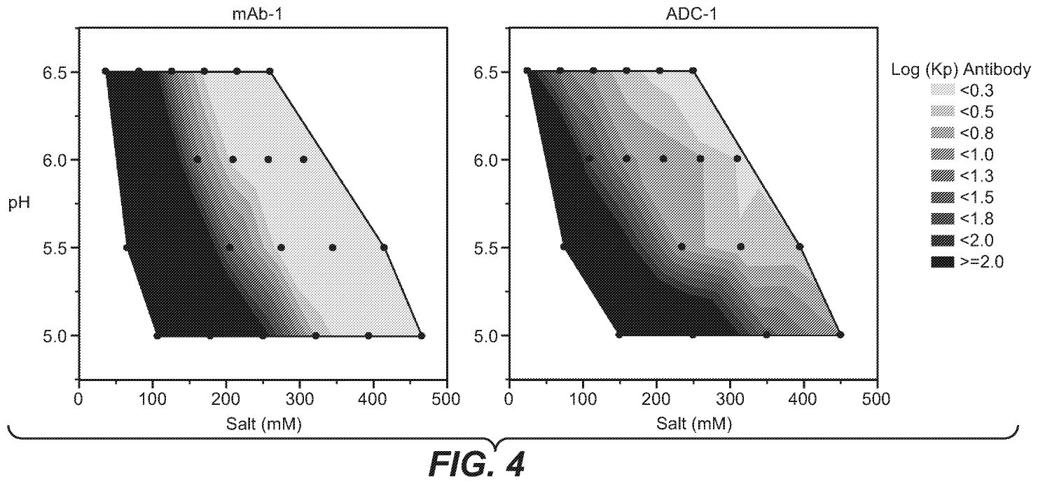

To determine flow-through conditions for ADC-1, a manual batch-binding screening was performed with the CEX resin using multichannel pipettes and 96-well plates. The conjugate screening results were comparable between the antibody and conjugate with similar binding behavior (FIG. 4). Load conditions of 214 mM sodium acetate, pH 5.5, corresponding to a Log K, value of approximately 1, were selected as the target conditions to allow the monomer to flow-through while the aggregate remains bound to the resin.

The selected target purification condition was applied to ADC-1 such that the purification step could be run in flow-through mode. The ADC load was titrated to the target conductivity and pH and was loaded onto a column to a load density of 500 g/Lᵣ. The column was washed with 10 CVs of equilibration buffer to recover the ADC-1. The load and pool were analyzed by SEC-HPLC for aggregates and HIC-HPLC for impact to DAR (Table 3).

TABLE 3
ADC-1 Purification Results

| | vHMWS | HMWS | Main | LMWS | Average |
|---|---|---|---|---|---|
| | (%) | (%) | (%) | (%) | DAR |
| Load | 0.74 | 2.42 | 96.2 | 0.69 | 3.8 |
| Pool | 0.10 | 1.92 | 97.3 | 0.64 | 3.8 |

The flow-through purification conditions were successful in reducing the vHMWS to 0.10% for ADC-1. A yield of 89% was achieved and there was no impact to average DAR or drug distribution. Although Antibody-1 did not use a step elution for purification, this study demonstrated that the antibody HTS could be leveraged for the ADC purification development and the manual batch-binding screening with the conjugate was unnecessary. Two additional products were evaluated to further test the theory that antibody HTS results can be leveraged to identify the flow-through conditions for ADC purification.

### Example 2 ADC-2 Purification Method

i. High-Throughput Screening for Antibody-2
a. Development of the Antibody-2 CEX step elution was performed using HTS. These experiments generated Log K, contour plots that supported identification of promising step elution conditions which were predicted to elute the monomer while the aggregate impurities remained bound to the resin. Based on these data, a target elution buffer was selected for Antibody-2, and subsequent robustness studies confirmed the robustness of the step elution purification.
   ii. ADC-2 Purification Development
b. The step elution conditions for Antibody-2 were leveraged during development of the ADC-2 CEX step to identify conditions so the aggregate would bind to the resin and be removed while the desired product (monomer) flows through. To achieve this, the column equilibration, load, and wash phases were adjusted to match the elution conditions (pH and conductivity) used for the Antibody-2 process.
c. After demonstrating successful aggregate removal at the target conditions, the process robustness around the target was tested at a range of pH and conductivity conditions (Table 4). The “Low” conditions were selected that represent stronger binding conditions for the CEX column due to decreased pH and decreased conductivity. Conversely, “High” load conditions were identified that would decrease binding of product but also likely decrease the removal of aggregate. Multiple “High” load conditions were tested with varying pH and conductivity ranges to investigate the robustness of the operation around the worst-case conditions in terms of aggregate removal. The conjugate load was adjusted to the desired conductivity and pH using the appropriate buffer species. The equilibration and wash buffers were adjusted to match the various conditions of the load. The load density ranged from 220-300 g/Lᵣ.

TABLE 4
ADC-2 Purification results over the five load conditions tested

| Load | Range from | | vHMWS | HMWS | Yield |
|---|---|---|---|---|---|
| Condition | target | | (%) | (%) | (%) |
| Low | −0.1 pH, −1.0 | Load | 0.61 | 0.73 | 81% |
| | mS/cm | Pool | 0.09 | 0.13 | |
| Target | N/A | Load | 0.66 | 0.78 | 95% |
| | | Pool | 0.01 | 0.57 | |
| High | +0.1 pH, +0.7 | Load | 0.80 | 0.80 | 97% |
| | mS/cm | Pool | 0.02 | 0.74 | |
| High+ | +0.2 pH, +0.7 | Load | 0.53 | 1.06 | 97% |
| | mS/cm | Pool | 0.03 | 1.07 | |
| High++ | +0.2 pH, +1.1 | Load | 0.57 | 0.79 | 98% |
| | mS/cm | Pool | 0.54 | 0.74 | |

d. The “Low” load conditions presented initial binding to resin and subsequent breakthrough, similar to overload mechanism of purification, with the HMWS also significantly reduced at this load condition (H. F. Liu, B. McCooey, T. Duarte, D. E. Myers, T. Hudson, A. Amanullah, R. van Reis, B. D. Kelley, Exploration of overloaded cation exchange chromatography for monoclonal antibody purification, J Chromatogr A. 1218 (2011) 6943-52). The yield was improved at a higher mass load density. The “High++” load conditions identified a point of failure and did not remove vHMWS. However, a range of load conditions were demonstrated as robust around the target load conditions between the “Low” to “High+” conditions (±0.1 pH, 0.7 mS/cm). The load and buffer specifications were set to ensure robust implementation into manufacturing.
e. After an acceptable operating range was demonstrated, the load density was challenged up to 500 g/Lᵣ using a 0.66 cm ID by 20 cm BH column. The conjugated pool was adjusted to the target load pH and conductivity and loaded on to the column to 500 g/Lₜ.
f. The yield from the high load density experiment was 96% and the vHMWS was reduced to 0.02% in the pool (Table 5). Additionally, there was no impact to average DAR with purification. The Antibody-2 CEX step elution conditions were effectively applied to the conjugate in flow-through mode at a load density of 500 g/Lᵣ.

TABLE 5
ADC-2 High Load Density Experiment Results

| | vHMWS | HMWS | Main | LMWS | Average |
|---|---|---|---|---|---|
| | (%) | (%) | (%) | (%) | DAR |
| Load | 0.69 | 0.77 | 98.5 | 0.04 | 1.8 |
| Pool | 0.02 | 0.68 | 99.2 | 0.10 | 1.8 |

The ADC-2 purification process was successfully scaled-up to manufacturing scale using a 14 cm ID by 15 cm BH column. Three runs at target conditions were performed, with the column loaded to approximately 260 g/Lᵣ per run. The drug substance results showed no detectable vHMWS and met all other product quality attributes (Table 6). Additionally, the chromatograms for the three runs were consistent and there was no increase in pressure during the load phase.

TABLE 6
ADC-2 Manufacturing Scale Purification
Results at Target Conditions

| | vHMWS | HMWS | Step Yield |
|---|---|---|---|
| Run | (%)^{a} | (%)^{a} | (%) |
| 1 | ND | 0.3 | 94 |
| 2 | ND | 0.3 | 94 |
| 3 | ND | 0.3 | 95 |

^{a}Abbreviations: ND = not detected.
^{a}Results are for the final drug substance. In-process pool analysis was not performed for large scale runs.

### Example 3: ADC-3 Purification Development

Similar to Antibody-2, the purification process for Antibody-3 employed a step elution CEX step to remove aggregates and impurities. Without performing any ADC purification development, the Antibody-3 step elution conditions were leveraged for the ADC-3 purification. The antibody step elution conditions were applied to the ADC such that the aggregate would bind to the resin and be removed while the desired product (monomer) flows through.

In order to achieve product flow-through, the conjugated ADC-3 pH and conductivity were adjusted to the target Antibody-3 elution buffer conditions. The adjusted conjugate load was loaded onto the CEX column to a load density of 500 g/Lᵣ. The column was washed with 10 CVs of the equilibration buffer and fractions were collected every 50 g/Lᵣ. Fractions were pooled and the results were compared to the load (Table 7).

TABLE 7
ADC-3 Purification Results

| | vHMWS | HMWS | Main | LMWS | Average |
|---|---|---|---|---|---|
| | (%) | (%) | (%) | (%) | DAR |
| Load | 0.39 | 0.43 | 98.88 | 0.29 | 3.4 |
| Pool | 0.03 | 0.41 | 99.23 | 0.33 | 3.4 |

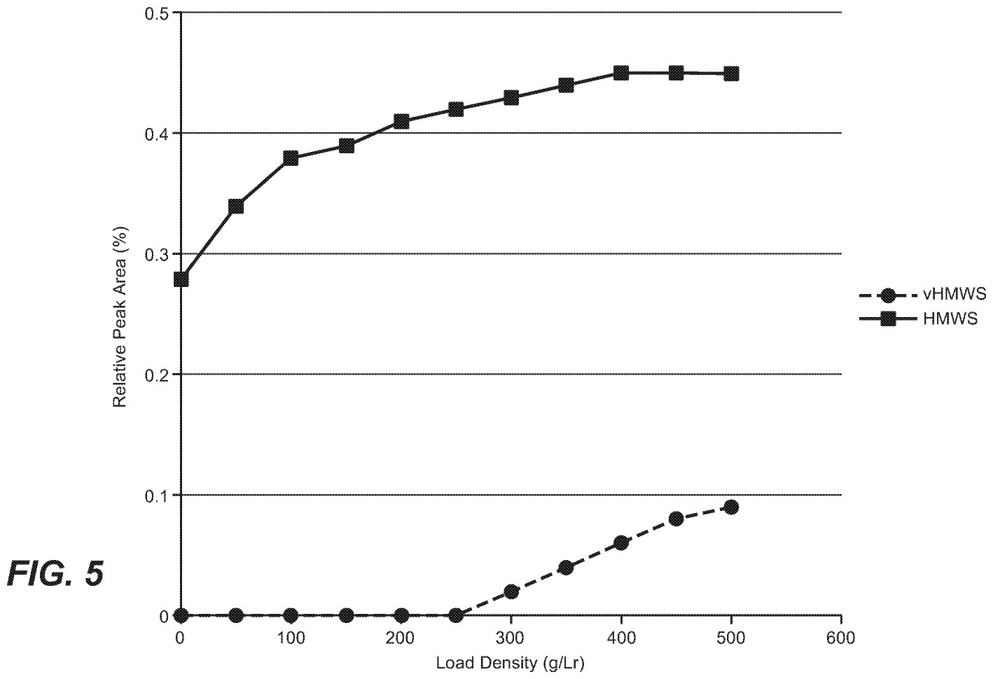

A small breakthrough of the vHMWS was observed starting at 300 g/Lᵣ load density (FIG. 5). To mitigate against vHMWS breakthrough, a lower load density or slightly stronger binding conditions were used. However, the final level of vHMWS in the purified pool was reduced to an acceptable level even when challenged to 500 g/Lᵣ load density.

These results demonstrate that the antibody intermediate purification step can be leveraged for ADC purification. No ADC purification development was performed as the Antibody-3 step elution conditions were successfully implemented for the conjugate to be run in flow-through mode. The flow-through purification conditions were successful in reducing the vHMWS from 0.39% to 0.03% for ADC-3 with a yield of 98%. Additionally, there was no impact to the average DAR and drug distribution.

### CONCLUSIONS

The antibody purification development data can be used to streamline development of a simple, flow-through purification step for their respective conjugates. The antibody step elution conditions (pH and conductivity) can be translated to the ADC to allow flow-through purification. In the absence of antibody step elution conditions, antibody HTS can be leveraged to identify ideal ADC flow-through conditions. The ADC purification steps:
significantly reduced the vHMWS
consistently achieved high yields
did not change to the average DAR or drug load distribution

The disclosure set forth above may encompass multiple distinct inventions with independent utility. Numerous variations are possible and the specific embodiments thereof as disclosed and illustrated herein are not to be considered in a limiting sense. The following claims particularly point out certain combinations and sub combinations regarded as novel and nonobvious. Inventions embodied in other combinations and sub combinations of features, functions, elements, and/or properties may be claimed in applications claiming priority from this or a related application. Such claims, whether directed to a different invention or to the same invention, and whether broader, narrower, equal, or different in scope to the original claims are regarded as included within the subject matter of the inventions taught herein.

We claim:

1. A method for reducing the concentration of protein aggregates in a cysteine-directed antibody drug conjugate (cys ADC), the method comprising the steps;
a. performing a first purification of an antibody with cation exchange chromatography material using a first set of purification condition to obtain a purified antibody intermediate;
b. conjugating the said purified antibody intermediate with a cytotoxic agent to form a crude preparation comprising of cys ADC and protein aggregates; and
c. performing a second purification of said crude with cation exchange chromatography material in flow-through mode using said first set of purification condition to generate a purified cys ADC,
wherein the said set of purification condition comprises load density, buffer species, pH and conductivity of the buffer systems.

2. The method of claim 1 further comprising an additional step of washing the cation exchange chromatography material to recover purified cys ADC.

3. The method of claim 2 wherein the yield of the purified cys ADC is above 98% w/w.

4. The method of claim 2 wherein the concentration of protein aggregates in the purified cys ADC is reduced by at least 85% relative to the concentration of protein aggregates in the crude mixture of cys ADC and protein aggregates without a change to critical quality attributes (CQA) of the cys ADC.

5. The method of claim 1 wherein the protein aggregates are selected from a group consisting of very high molecular weight species (vHMWS) and high molecular weight species (HMWS).

6. The method of claim 5 wherein the protein aggregates are vHMWS.

7. The method of claim 6 wherein the vHMWS is an oligomer.

8. The method of claim 2 wherein the vHMWS in the purified cys ADC is reduced to less than 0.1%.

9. The method of claim 2 wherein the vHMWS in the cys ADC is reduced to less than 0.02%.

10. A method of purifying cysteine-directed antibody drug conjugate (cys ADC), the method comprising the steps;
a. performing a first purification of an antibody with cation exchange chromatography material using a first set of purification condition to obtain a purified antibody intermediate;
b. conjugating the said purified antibody intermediate with a cytotoxic agent to form a crude preparation of cys ADC; and
c. performing a second purification of said crude preparation with cation exchange chromatography material in flow-through mode using said first set of purification condition to generate a purified cys ADC,
wherein the said set of purification condition comprises load density, buffer species, pH and conductivity of the buffer systems.

11. The method of claim 10 further comprising an additional step of washing the cation exchange chromatography material to recover purified cys ADC.

12. The method of claim 11 wherein the yield of the purified cys ADC is above 98% w/w.

13. The method of claim 1 or claim 10 wherein the cys ADC is selected from a group consisting of site-specific conjugate via an engineered cysteine and interchain-cysteine conjugate that target native cysteines.

14. The method of claim 13 wherein the cys ADC is site-specific conjugate via an engineered cysteine.

15. The method of claim 13 wherein the cys ADC is interchain-cysteine conjugate that target native cysteines.

16. The method of claim 1 or claim 10 wherein the cation exchange material is a resin.

17. The method of claim 16 wherein the resin is selected from a group consisting of POROS 50HS, POROS XS, and SPFF resins.

18. The method of claim 17 wherein the resin is POROS XS.

19. The method of claim 17 wherein the resin is SPFF.

20. The method of claim 1 or claim 10 wherein the load density in the second purification of the crude mixture of cys ADC and protein aggregates is from 100 g/Lᵣ to 1000 g/Lᵣ.

21. The method of claim 20 wherein the load density in the second purification of the crude mixture of cys ADC and protein aggregates is 500 g/Lᵣ.

22. The method of claim 1 or claim 10 wherein the first purification of the antibody using a first set of purification condition to obtain a purified antibody intermediate is performed in a bind-elute mode.

23. The method of claim 1 or claim 10 wherein the cytotoxic agent is selected from a group consisting of Auristatins, Maytansinoids, and DNA-damaging agents.

24. The method of claim 23 wherein the DNA-damaging agent is a derivative selected from a group consisting of Calicheamicin, Anthracyclines, and Pyrrolobenzodiazepines.

25. The method of claim 24 wherein the cytotoxic agent is a Pyrrolobenzodiazepine derivative.

26. The method of claim 25 wherein the cytotoxic agent is Pyrrolobenzodiazepine monoamide.

27. The method of claim 23 wherein the cytotoxic agent is a derivative of Auristatin.

28. The method of claim 27 wherein the cytotoxic agent is MMAE (Monomethyl Auristatin E).

29. The method of claim 1 or claim 10 wherein the cytotoxic agent forms a linker drug complex.

30. The method of claim 29 wherein the linker drug complex is vcMMAE (Monomethyl Auristatin E, cytotoxin with valine-citrulline (vc-) linker).
